(19) United States
(12) Patent Application Publication
(10) Pub. No.: US 2017/0224606 A1
(43) Pub. Date: Aug. 10, 2017

(54) POLYURETHANE UREA SOLUTIONS FOR HAIR-STYLING COMPOSITIONS

(71) Applicant: Covestro Deutschland AG, Leverkusen (DE)

(72) Inventors: Sophie Viala, Köln (DE); Sebastian Dörr, Düsseldorf (DE); Paula Cristina Alves Rodrigues, Düsseldorf (DE)

(21) Appl. No.: 15/501,290

(22) PCT Filed: Mar. 26, 2015
(86) PCT No.: PCT/EP2015/056509

§ 371 Date: Feb. 2, 2017

(30) Foreign Application Priority Data
Aug. 5, 2014 (EP) 14179783.7

(51) Int. Cl. A61K 8/87; A61K 8/31; C08G 18/75; A61K 8/34; C08G 18/10; C08G 18/66; A61K 8/04; A61Q 5/06

(52) U.S. Cl. CPC A61K 8/87; A61K 8/046; A61K 8/31; A61Q 5/06; A61K 8/34; C08G 18/10; C08G 18/6685; C08G 18/755; A61K 2800/22; A61K 2800/48

(57) ABSTRACT

The invention relates to a hair-styling composition, comprising at least one propellant gas and/or a thickener, and a polyurethane urea, which has no ionic hydrophilizing groups and which is dissolved in a solvent or solvent mixture, the solvent consisting of one or more monohydroxy-functional alcohols or a solvent mixture consisting of organic solvents, which solvent mixture contains ≧50 wt. % of at least one monohydroxy-functional alcohol, with respect to the total mass of the solvent mixture. The invention further relates to the use of the hair-styling composition to shape, solidify, and/or fix hair to a method for shaping, solidifying, and/or fixing hair by using the polyurethane urea solution. The invention further relates to a method for producing a hair-styling composition, in which method the polyurethane solution is used.

### CROSS-REFERENCE TO RELATED APPLICATIONS

This application is a National Phase Application of PCT/EP2015/056509, filed Mar. 26, 2015, which claims priority to European Application No. 14179783.7, filed Aug. 5, 2014, both of which are incorporated herein by reference.

### FIELD OF THE INVENTION

The present invention relates to a hair-styling composition comprising at least one thickener and/or a propellant gas and a specific polyurethane urea, and to the use of the hair-styling compositions for shaping, setting and/or fixing of hair, and to a method of shaping, setting and/or fixing hair using the specific polyurethane urea. The invention further provides a process for producing a hair-styling composition, wherein a specific polyurethane urea is used, and to a polyurethane urea obtainable by the process of the invention.

### BACKGROUND OF THE INVENTION

Hair-styling products are often in the form of preparations sprayable from aerosol containers or squeeze bottles or by means of pumping, spraying or foaming devices, and these predominantly consist of an alcoholic solution of film-forming natural or synthetic polymers.

Known good film-forming polymers for hair-styling compositions include aqueous dispersions of polyurethane ureas, as described, for example, in WO 2009/118105 A1, WO 2012/130683 A1 and WO 2012/130682 A1. Hair-styling compositions comprising the aqueous polyurethane urea dispersions described have some advantages, such as good fixing of hairdos. However, the known aqueous dispersions of the polyurethane ureas have some disadvantages in hair-styling compositions based predominantly on alcoholic solvents. For instance, the film formers described in WO2009/118105 A1, WO 2012/130683 A1, WO 2012/130682 A1 and WO 2014/095164 A1 form a clear mixture with alcohol/water mixtures only, but are incompatible with pure ethanol, for example, and turn the composition cloudy. This is perceived as being troublesome for many applications. Another disadvantage is that the polyurethane ureas according to prior art have hydrophilizing groups, especially ionically hydrophilizing groups, which are introduced into the polymers by means of costly compounds that bear these groups.

Polyurethane ureas that bear ionically hydrophilizing groups additionally do not generally form clear solutions in alcohols, which mean that they are not very suitable for use in transparent cosmetic compositions.

It is likewise known that the polyurethane-based film formers known from the state of the art have poorer compatibility with propellant gas mixtures, for example propane/butane, which are used in aerosol formulations.

Polyurethane film formers that bear hydrophilizing groups, especially ionically hydrophilizing groups, are also described, for example, in DE 4241118 A1.

It is common knowledge that polyurethane ureas, because of their structure, tend to precipitate or crystallize out of organic solutions. It is therefore problematic to produce organic solutions of polyurethane ureas having a sufficiently high molecular weight without precipitation of the polyurethane ureas out of the solvents, and therefore no clear, storage-stable solutions are obtained.

For prevention of this crystallization, solvent mixtures comprising solvents which are now counted among the potentially harmful solvents on the basis of growing toxicological knowledge are recommended, for example toluene or xylene.

However, the use of such co-solvents for polyurethane ureas for use in cosmetic products is not possible merely for approval-related legal reasons.

### SUMMARY OF THE INVENTION

The present invention provides a transparent alcohol-based hair-styling composition based on polyurethane ureas as film formers, wherein the film formers have good compatibility with propellant mixtures such as propane/butane and hence have good suitability for aerosol formulations. Moreover, the compositions were to bring about good setting of the hair.

It is understood that the invention disclosed and described in this specification is not limited to the embodiments summarized in this Summary.

### BRIEF DESCRIPTION OF THE FIGURES

The present invention will now be described for purposes of illustration and not limitation in conjunction with the figures, wherein:

FIG. 1 shows a graph of the measurement of curl retention versus time; and

FIG. 2 depicts a bar chart of the curl recovery with various formulations at three different combing times; once, five and ten times.

### DETAILED DESCRIPTION OF THE INVENTION

The present invention will now be described for purposes of illustration and not limitation.

The present invention provides a hair-styling composition comprising at least one propellant gas and/or a thickener, characterized in that it further comprises a polyurethane urea which has no ionically hydrophilizing groups and has been dissolved in a solvent or solvent mixture, the solvent consisting of one or more monohydroxy-functional alcohols or a solvent mixture consisting of organic solvents and containing ≧50% by weight, based on the total mass of the solvent mixture, of at least one monohydroxy-functional alcohol, being used. The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The dissolved polyurethane urea used in accordance with the invention, including the solvent or solvent mixture, is also referred to hereinafter as polyurethane urea solution. It has been found that, surprisingly, through the use of polyurethane urea solutions in monohydroxy-functional alcohols or solvent mixtures of organic solvents and monohydroxy-functional alcohols, it is possible to obtain transparent alcohol-based hairsetting compositions, wherein the film formers have good compatibility with propellant mixtures such as propane/butane and hence are of good suitability for aerosol formulations. Moreover, the compositions bring about good fixing of the hair.

The present invention further provides a process for producing a hair-styling composition, characterized in that at least one polyurethane urea which has no ionically hydrophilizing groups and has been dissolved in a solvent or solvent mixture is used, the solvent consisting of one or more monohydroxy-functional alcohols or being a solvent mixture consisting of organic solvents and containing ≧50% by weight, based on the total mass of the solvent mixture, of at least one monohydroxy-functional alcohol.

The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The invention likewise provides the hair-styling composition obtainable by the process of the invention.

The invention further provides for the use of a polyurethane urea which has no ionically hydrophilizing groups and has been dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol, for production of a hair-styling composition. The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The invention likewise provides for the use of a polyurethane urea which has no ionically hydrophilizing groups and has been dissolved in a solvent or solvent mixture, wherein the solvent consists of one or more monohydroxy-functional alcohols or a solvent mixture consisting of organic solvents and containing ≧50% by weight, based on the total mass of the solvent mixture, of at least one monohydroxy-functional alcohol, for shaping, setting and/or fixing of hair. The polyurethane urea used is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

“Dissolved” in the context of the invention means clear liquid mixtures of at least two substances that are homogeneous and monophasic at 23° C. “Clear” in the context of the present invention means that the turbidity values of the solution are ≦200 NTU (Nephelometric Turbidity Unit), preferably ≦50 NTU, more preferably ≦10 NTU and most preferably ≦5 NTU. Turbidity values are determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

Polyurethane ureas in the context of the invention are polymeric compounds having at least two, preferably at least three, urethane-containing repeat units

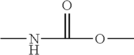

and additionally also urea-containing repeat units:

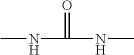

Ionically hydrophilizing groups in the context of the invention are those which could be introduced into the polyurethane urea, for example, by means of suitable anionically or potentially anionically hydrophilizing compounds having at least one isocyanate-reactive group, such as a hydroxyl or amino group, and at least one functionality, for example, —COO-M⁺, —SO₃-M⁺, —PO(O-M⁺)₂ where M⁺, for example is a metal cation, H⁺, NH₄⁺, NHR₃⁺ where each R is a C₁-C₁₂-alkyl radical, C₅-C₆-cycloalkyl radical and/or a C₂-C₄-hydroxyalkyl radical, which enters into a pH-dependent dissociation equilibrium on interaction with aqueous media and in this way may be negatively charged or uncharged. Suitable anionically or potentially anionically hydrophilizing compounds are mono- and dihydroxycarboxylic acids, mono- and dihydroxysulfonic acids, and mono- and dihydroxyphosphonic acids and salts thereof. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as described in DE-A 2 446 440, pages 5-9, formula I-III.

Potentially anionic (and also generally potentially ionic) groups in the context of this invention are understood to mean those which can be converted by neutralization to an anionic (ionic) group.

In a preferred embodiment of the process of the invention, the polyurethane urea used does not have any hydrophilizing groups, i.e. neither ionic nor nonionic hydrophilizing groups.

Nonionic hydrophilizing groups in the context of the invention are those which could be introduced into the polyurethane urea, for example, by means of suitable nonionically hydrophilizing compounds, for example polyoxyalkylene ethers containing at least one hydroxyl or amino group. Examples are the monohydroxy-functional polyalkylene oxide polyether alcohols having a statistical average of 5 to 70, preferably 7 to 55, ethylene oxide units per molecule, as obtainable by alkoxylation of suitable starter molecules (described, for example, in Ullmanns Encyclopadie der technischen Chemie [Ullmann's Encyclopedia of Industrial Chemistry], 4th edition, volume 19, Verlag Chemie, Weinheim p. 31-38). These compounds are either pure polyethylene oxide ethers or mixed polyalkylene oxide ethers, in which case, however, they contain at least 30 mol %, preferably at least 40 mol %, based on all alkylene oxide units present, of ethylene oxide units.

The polyurethane ureas of the present invention are used in the hair-styling compositions of the invention and in the process of the invention for producing the cosmetic compositions in dissolved form in a solvent or solvent mixture, and hence as polyurethane urea solutions and not as an aqueous dispersion.

The polyurethane urea used in accordance with the invention is formed from
a) at least one of an aliphatic, an araliphatic and a cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

The number-average molecular weight is always determined in the context of this application by gel permeation chromatography (GPC) against polystyrene standard in tetrahydrofuran at 23° C. The procedure is according to DIN 55672-1: “Gel permeation chromatography, Part 1—Tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used for calibration. The number-average molecular weight is calculated with software support. Baseline points and evaluation limits are fixed in accordance with DIN 55672 Part 1.

Further preferably, the polyurethane urea is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Compounds suitable as component a) are, for example, butylene 1,4-diisocyanate, pentamethylene 1,5-diisocyanate (PDI), hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any isomer content (H12-MDI), cyclohexylene 1,4-diisocyanate, 4-isocyanatomethyloctane 1,8-diisocyanate (nonane triisocyanate), 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) having C₁-C₈-alkyl groups.

As well as the aforementioned polyisocyanates, it is also possible to use proportions of modified diisocyanates or triisocyanates having isocyanurate, urethane, allophanate, biuret, iminooxadiazinedione and/or oxadiazinetrione structure.

Preferably, the polyisocyanates or polyisocyanate mixtures are of the aforementioned type with a mean NCO functionality of ≧2 to ≦4, preferably of ≧2 to 2.6 and more preferably of ≧2 to ≦2.4.

Preferably, component a) is selected from aliphatic, araliphatic and/or cycloaliphatic diisocyanates having at least one isocyanate group bonded to a secondary and/or tertiary carbon atom.

More preferably, component a) is selected from IPDI and/or H12-MDI.

Further preferably, no aromatic polyisocyanates are used for preparation of the polyurethane urea.

Component a) is preferably used in amounts of ≧5% and ≦60% by weight, more preferably ≧10% and ≦40% by weight and most preferably of ≧15% and ≦35% by weight, based on the total weight of the polyurethane ureas.

Component b) comprises one or more polyether polyols having a number-average molecular weight Mn ≧400 and ≦6000 g/mol and a hydroxyl functionality of ≧1.5 and ≧4, preferably having a number-average molecular weight Mn ≧500 and ≦2500 g/mol and a hydroxyl functionality of ≧1.9 and ≦3 and more preferably having a number-average molecular weight Mn ≧1000 and ≦2000 g/mol and a hydroxyl functionality of ≧1.9 and ≦2.1.

Suitable polyether polyols of component b) are, for example, the poly(tetramethylene glycol) polyether polyols known in polyurethane chemistry, as obtainable by polymerization of tetrahydrofuran by means of cationic ring opening.

Likewise suitable polyether polyols are the addition products of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are applicable, especially with the abovementioned preferred molecular weights. The polyether polyols preferably have a proportion of groups obtained from ethylene oxide of ≦50% by weight, preferably ≦30% by weight.

Suitable starter molecules used may be all compounds known in the art, for example water, butyldiglycol, glycerol, diethylene glycol, trimethylolpropane, propylene glycol, sorbitol, ethylenediamine, triethanolamine, butane-1,4-diol.

Preferably, component b) are selected from polypropylene glycols and/or poly(tetramethylene glycol) polyether polyols, more preferably selected from poly(tetramethylene glycol) polyether polyols.

In a preferred employment of the invention, component b) comprises one or more poly(tetramethylene glycol) polyether polyols having a number-average molecular weight Mₙ ≧500 and ≦2500 g/mol and a hydroxyl functionality of ≧1.9 and ≦2.1.

In a particularly preferred embodiment, component b) is a mixture of poly(tetramethylene glycol) polyether polyols I having a number-average molecular weight Mₙ of ≧400 and ≦1500 g/mol, more preferably of ≧600 and ≦1200 g/mol, most preferably of 1000 g/mol, and poly(tetramethylene glycol) polyether polyols II having a number-average molecular weight Mₙ of ≧1500 and ≦8000 g/mol, more preferably of ≧1800 and ≦3000 g/mol, most preferably of 2000 g/mol.

The weight ratio of the poly(tetramethylene glycol) polyether polyols I to the poly(tetramethylene glycol) polyether polyols II is preferably in the range of ≧0.1 and ≦10, more preferably in the range of ≧0.2 and ≦8, most preferably in the range of ≧1 and ≦6.

Component b) is preferably used in amounts of ≧30% and ≦90% by weight, more preferably ≧50% and ≦85% by weight, most preferably of ≧55% and ≦75% by weight, based on the total weight of the polyurethane urea.

Component c) is one or more amino-functional compounds having at least two isocyanate-reactive groups.

Suitable components c) are, for example, di- or polyamines such as ethylene-1,2-diamine, 1,2- and 1,3-diaminopropane, 1,4-diaminobutane, 1,6-diaminohexane, 2,2,4- and 2,4,4-trimethylhexamethylenediamine, 2-methylpentamethylenediamine, diethylenetriamine, triaminononane, xylylene-1,3- and 1,4-diamine, α,α,α′,α′-tetramethylxylylene-1,3- and -1,4-diamine and 4,4′-diaminodicyclohexylmethane (H12-MDA), isophoronediamine (IPDA) and/or 1,2-dimethylethylenediamine.

Preferably, component c) is selected from ethyleneamine, IPDA and/or H12-MDA, more preferably from isophoronediamine and/or H12-MDA, and component c) is most preferably H12-MDA.

The compounds of component c) preferably do not contain any hydrophilizing groups, and more particularly no ionically or potentially anionically hydrophilizing groups. In a particularly preferred embodiment of the invention, component c) is selected from amines having at least two isocyanate-reactive amino groups bonded to primary and/or secondary carbon atoms.

Further preferably, component c) is selected from diamines of symmetric structure.

Most preferably, component c) is selected from symmetric diamines having at least two amino groups bonded to primary and/or secondary carbon atoms; component c) is especially preferably H12-MDA.

Component c) is preferably used in amounts of ≧2% and ≦25% by weight, more preferably ≧5% and ≦20% by weight and most preferably ≧9% and ≦16% by weight, based on the total weight of the polyurethane urea.

In a preferred embodiment of the invention, either component a) is H12-MDI or component c) is H12-MDA or component a) is H12-MDI and component c) is H12-MDA.

Optionally, the polyurethane urea is additionally formed from component d), one or more alcohols having at least two hydroxyl groups and a molar mass of ≧60 and ≦399 g/mol, for example polyols of the molar mass range mentioned having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxyphenyl)propane), trimethylolpropane, glycerol, pentaerythritol.

Component d) is preferably used in amounts of ≧0% and ≦10% by weight, more preferably ≧0% and ≦3% by weight, based on the total weight of the polyurethane urea, and is most preferably not used at all.

In addition, the polyurethane ureas may be formed from component e), one or more compounds having a group reactive toward isocyanate groups, especially compounds having an amino or hydroxyl group. Suitable compounds of component e) are, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, methanol, ethanol, isopropanol, n-propanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Component e) preferably does not comprise any monofunctional polyether polyols having a proportion of groups obtained from ethylene oxide of >30% by weight, preferably >50% by weight.

The monohydroxy-functional alcohol used as solvent for the polyurethane urea can likewise serve as formation component e) for the polyurethane urea.

Component e) is used preferably in amounts of ≧0% and ≦10% by weight, more preferably ≧0% and ≦3% by weight, based on the total weight of the polyurethane urea, and is most preferably not used at all, not including the monohydroxy-functional alcohol used as solvent for the polyurethane urea as component e).

The monohydroxy-functional alcohol which serves as solvent for the polyurethane urea makes up preferably ≧0% and ≦5% by weight, more preferably ≧0.01% and ≦3% by weight and most preferably ≧0.01% and ≦2% by weight of the total mass of the polyurethane urea.

The polyurethane urea may also be formed from component f), a polyol or two or more polyols having a number average molecular weight Mₙ of ≧500 and ≦6000 g/mol and the hydroxyl functionality of ≧1.5 and ≦4, the polyols being different than b).

Component f) is preferably used in amounts of ≧0% and ≦20% by weight, more preferably ≧0% and ≦10% by weight, based on the total weight of the polyurethane urea, and is most preferably not used at all.

Preferably, the polyols of component f) have a number-average molecular weight Mₙ of ≧1000 and ≦3000 g/mol and a hydroxyl functionality of ≧1.8 and ≦3.

Polyols suitable as component f) are the following polyols that are known in polyurethane coating technology: polyester polyols, polyacrylate polyols, polyurethane polyols, polycarbonate polyols, polyester polyacrylate polyols, polyurethane polyacrylate polyols, polyurethane polyester polyols, polyurethane polyether polyols, polyurethane polycarbonate polyols, polyether polycarbonate polyols and/or polyester polycarbonate polyols, especially polyester polyols and/or polycarbonate polyols.

Polyester polyols are, for example, the polycondensates of di- and optionally tri- and tetraols, and optionally tri- and tetracarboxylic acids or hydroxycarboxylic acids or lactones. Instead of the free polycarboxylic acids, it is also possible to use the corresponding polycarboxylic anhydrides or corresponding polycarboxylic esters of lower alcohols to produce the polyesters.

Examples of diols suitable for this purpose are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate, preference being given to hexane-1,6-diol and isomers, neopentyl glycol and neopentyl glycol hydroxypivalate. In addition, it is also possible to use polyols such as trimethylolpropane, glycerol, erythritol, pentaerythritol, trimethylolbenzene or tris-hydroxyethyl isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible to use the corresponding anhydrides as acid source.

If the mean hydroxyl functionality of the polyol to be esterified is greater than 2, it is additionally also possible to use monocarboxylic acids such as benzoic acid and hexanecarboxylic acid as well.

Preferred acids are aliphatic or aromatic acids of the aforementioned type. Particular preference is given to adipic acid, isophthalic acid and optionally trimellitic acid, very particular preference to adipic acid.

Examples of hydroxycarboxylic acids that may be used as reaction participants in the preparation of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologues. Preference is given to caprolactone.

In component f), it is also possible to use polycarbonates having hydroxyl groups, preferably polycarbonatediols, having number-average molecular weights Mₙ of 400 to 8000 g/mol, preferably of 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the aforementioned type. The polycarbonates having hydroxyl groups preferably have a linear structure.

In a preferred embodiment of the invention, the polyurethane urea used in accordance with the invention is formed from
a) at least one of an aliphatic, an araliphatic and a cycloaliphatic diisocyanate having at least one isocyanate group bonded to a secondary or tertiary carbon atom,
b) at least one polyether polyol having a number-average molecular weight Mₙ of ≧500 and ≦2500 g/mol and a hydroxyl functionality of ≧1.9 and ≦3,
c) at least one amino-functional compound having at least two (isocyanate-reactive) amino groups and selected from ethylenediamine, IPDA and/or H12-MDA,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

In a particularly preferred embodiment of the invention, the polyurethane urea used in accordance with the invention is formed from
a) at least one isocyanate selected from IPDI and H12-MDI,
b) at least one polyether polyol having a number average molecular weight Mₙ ≧500 and ≦2500 and a hydroxyl functionality of ≧1.9 and ≦3, selected from polypropylene glycols and/or poly(tetramethylene glycol) polyether polyols,
c) at least one amino-functional compound selected from IPDA and/or H12-MDA,
d) optionally, at least one alcohol having at least two hydroxyl groups and a molar mass of ≧60 and ≦399 g/mol,
e) optionally, at least one compound having a group reactive toward isocyanate groups and
f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

Further preferably, the polyurethane urea, in this aforementioned embodiment, is formed from ≧5% and ≦60% by weight of component a), ≧30% and ≦90% by weight of component b), ≧2% and ≦25% by weight of component c), ≧0% and ≦10% by weight of component d), ≧0% and ≦10% by weight of component e) and ≧0% and ≦20% by weight of component f), based in each case on the total mass of the polyurethane urea, where components a) to f) add up to 100% by weight.

Especially preferably, the polyurethane urea, in this aforementioned embodiment, is formed from ≧10% and ≦40% by weight of component a), ≧55% and ≦85% by weight of component b), ≧5% and ≦20% by weight of component c), ≧0% and ≦3% by weight of component d), ≧0% and ≦3% by weight of component e) and ≧0% and ≦1% by weight of component f), based in each case on the total mass of the polyurethane urea, where components a) to f) add up to 100% by weight.

Preferably, the polyurethane urea is formed from components a) to c) and optionally d) to f), more preferably from components a) to c).

Advantageously, the polyurethane urea has a number-average molecular weight Mₙ ≧2000 and ≦50 000 g/mol, particularly advantageously ≧3000 and ≦30 000 g/mol.

The polyurethane urea is preferably prepared by reacting components a) and b) and optionally d) and f) in a first step to give an NCO-terminated prepolymer, which is then reacted in a subsequent step with component c) and optionally components d) and e).

For the preparation of the polyurethane ureas, preferably, components a) and b) and optionally d) and f) for preparation of an NCO-terminated prepolymer are initially charged in full or in part, optionally diluted with a solvent inert toward isocyanate groups, and heated up to temperatures in the range from 50 to 120° C. The isocyanate addition reaction can be accelerated using the catalysts known in polyurethane chemistry. A preferred variant, however, works without the addition of urethanization catalysts.

Subsequently, any constituents of a) and b) and optionally d) and f) which have not yet been added at the start of the reaction are metered in.

In the preparation of the NCO-terminated prepolymers from components a) and b) and optionally d) and f), the molar ratio of isocyanate groups to isocyanate reactive groups is generally ≧1.05 and ≦3.5, preferably ≧1.1 and ≦3.0, more preferably ≧1.1 and ≦2.5. Isocyanate-reactive groups are understood to mean all groups reactive toward isocyanate groups, for example primary and secondary amino groups, hydroxyl groups or thiol groups.

The conversion of components a) and b) and optionally d) and f) to the prepolymer is effected in part or in full, but preferably in full. In this way, polyurethane prepolymers containing free isocyanate groups are obtained in substance or in solution.

Preferably, the NCO-terminated prepolymer is prepared from components a) and b).

Thereafter, preferably, in a further process step, if this has been done only partly, if at all, the prepolymer obtained is dissolved with the aid of one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable. The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation consists of one or more monohydroxy-functionalized alcohols.

The ratio of solvent to prepolymer is preferably ≧1:10 and ≦5:1, more preferably ≧1:2 and ≦2:1, parts by weight.

Prior to the dissolution, the prepolymer is cooled down to temperatures of −20 to 60° C., preferably 0 to 50° C. and more preferably 15 to 40° C.

In a further step that optionally follows the dissolution of the NCO-terminated prepolymer, the NCO-terminated prepolymer obtained in the first step is then preferably reacted fully or partly with component c) and optionally components d) and e). This reaction is generally referred to as chain extension, or in the case of component e) as chain termination.

Preference is given here to initially charging the NCO-terminated prepolymer, and metering in components c) and optionally d) and e). Preference is given to firstly partly reacting the NCO groups of the prepolymer with components c) and optionally d), followed by chain termination by reaction of the remaining NCO groups with component e). Components c) and optionally e) may also be added stepwise in two or more steps, especially into steps.

Component c) and optionally d) and e) are preferably used dissolved in one or more organic solvents. The solvent used is preferably likewise a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used. In respect of the solvent and solvent mixture, the preferred embodiments below relating to the solvent or solvent mixture in which the polyurethane urea is dissolved are likewise applicable.

The solvent or solvent mixture may also be different than the solvent or solvent mixture in which the polyurethane urea as end product is dissolved at a later stage. The solvent or solvent mixture is preferably identical to the solvent or solvent mixture in which the polyurethane urea as end product is dissolved at a later stage.

Preferably, the solvent used in the preparation for component c) comprises one or more monohydroxy-functionalized alcohols.

When solvents are used as diluents, the diluent content in the components c) used in the chain extension, and optionally d) and e), is preferably 1% to 95% by weight, preferably 3% to 50% by weight, based on the total weight of component c) and optionally d) and e) including diluents.

Components c) and optionally d) and e) are preferably added at temperatures of −20 to 60° C., preferably 0 to 50° C. and more preferably of 15 to 40° C.

The degree of chain extension, i.e. the molar ratio of NCO-reactive groups of the components c) used for chain extension and chain termination, and optionally d) and e), to free NCO groups of the prepolymer, is generally ≧50 and ≦120%, more preferably ≧60 and ≦100% and most preferably ≧70 and ≦95%.

Preferably, the molar ratio of isocyanate-reactive groups of component c) to the free NCO groups of the prepolymer is ≧50% and ≦120%, more preferably ≧60% and ≦100% and most preferably ≧70% and ≦95%.

In a preferred embodiment of the invention, the free NCO groups of the prepolymer are only partly reacted with component c), the molar ratio of isocyanate-reactive groups of component c) to the free NCO groups of the prepolymer preferably being ≧60% and ≦95% and the remaining free NCO groups being depleted by reaction with the hydroxyl groups of the solvent, so as to form an NCO-free polyurethane urea.

After the preparation, the polyurethane urea, if solvents or solvent mixtures of the invention have already been used in the preparation process, can still be diluted and dissolved with a solvent or solvent mixture, in which case the solvent consists of one or more monohydroxy-functional alcohols or a solvent mixture consisting of organic solvents containing ≧50% by weight, based on the total mass of the solvent mixture, of at least one monohydroxy-functional alcohol is used.

If no solvents or solvent mixtures have been used during the reaction, after the polyurethane urea has been prepared, it is used in a solvent or solvent mixture, in which case the solvent consists of one or more monohydroxy-functional alcohols or a solvent mixture consisting of organic solvents and containing ≧50% by weight, based on the total mass of the solvent mixture, of at least one monohydroxy-functional alcohol is used.

The dissolution of the polyurethane urea can be effected by standard techniques for shearing, for example by stirring with standard stirrers as specified in DIN 28131.

The polyurethane urea is preferably dissolved without the additional addition of external emulsifiers. The polyurethane urea solutions used in accordance with the invention preferably do not comprise any external emulsifiers.

Suitable solvents or constituents of the solvent mixture are in principle all monohydroxy-functional aliphatic alcohols having one to six carbon atoms, for example methanol, ethanol, n-propanol, isopropanol, n-butanol, sec-butanol and/or butylglycol. More preferably, the monohydroxy-functional alcohol is ethanol.

If a solvent mixture is used, as well as the monohydroxy-functional alcohols, it is also possible to use ≦50% by weight, based on the total weight of the solvent mixture, of a further organic solvent. Suitable solvents here are, for example, esters, for example ethyl acetate, butyl acetate, methoxypropyl acetate or butyrolactone, ketones, for example acetone or methyl ethyl ketone, ethers, for example tetrahydrofuran or tert-butyl methyl ether, aromatic solvents, for example xylene or solvent naphtha. In the case of use of ethanol, typical denaturing agents may be present as additives in the customary added amounts.

Preferably, the proportion of the further organic solvents is ≦30% by weight, more preferably ≦5% by weight and most preferably ≦2% by weight, based on the total weight of the solvent mixture. In a most preferred embodiment, no further organic solvents are present aside from monohydroxy-functional aliphatic alcohols.

Unsuitable further solvents are physiologically incompatible solvents, for example dimethylformamide, N-methylpyrrolidone or toluene, as often used as cosolvents for polyurethanes or polyurethane ureas, these should preferably not be present in cosmetic compositions.

The further solvents are not water. The polyurethane urea solution obtained by dissolving the polyurethane urea in the solvents or solvent mixture is used in accordance with the invention is preferably anhydrous, excluding the proportions of water present as a result of the preparation in the organic solvents used.

The water content of the polyurethane urea solution is ≦10% by weight, preferably ≦4.5% by weight and most preferably ≦1% by weight, based on the total mass of the polyurethane urea solution.

The proportion of the polyurethane urea (as active substance) in the polyurethane urea solution used in accordance with the invention (also referred to as solids content) is preferably ≧10% and ≦80% by weight, more preferably ≧15% and ≦60% by weight and most preferably ≧20% and ≦50% by weight, based on the total weight of the polyurethane urea solution.

Preferably, the hair-styling compositions are those that are predominantly alcohol-based, i.e. contain ≧10% and ≦90% by weight, based on the total mass of the hair-styling composition, preferably ≧15% and ≦70% by weight and more preferably ≧20% and ≦60% by weight of aliphatic alcohols having 1 to 6 and preferably 1 to 4 carbon atoms. The alcohols are preferably selected from ethanol and isopropanol; polyol and derivatives thereof, such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, or mixtures thereof. More preferably, the alcohols contain ethanol or consist thereof; most preferably, the alcohol used is ethanol.

The hairsetting composition may also comprise further cosmetically suitable solvents, for example acetone, unbranched or branched hydrocarbons, cyclic hydrocarbons and/or mixtures thereof.

More preferably, the hair-styling compositions are alcoholic solutions.

The hairstyling compositions preferably contain a water content of ≧0% and ≦30% by weight, more preferably ≧0% and ≦20% by weight, even more preferably of ≧0% and ≦5% by weight and further preferably of ≧0% and ≦2% by weight. Especially preferably, the cosmetic compositions are anhydrous, and thus contain no more water than what is unavoidably introduced into the formulation via the raw materials as a result of production.

The proportion of the polyurethane urea solution used in the hair-styling composition is preferably ≧0.5% and ≦80% by weight, more preferably ≧1% and ≦60% by weight and most preferably ≧2% and ≦40% by weight, based on the total mass of the hairstyling composition.

The solids content of the polyurethane urea solution is preferably chosen such that the hair-styling compositions contain preferably ≧0.1% and ≦30% by weight, more preferably ≧0.2% and ≦20% by weight and most preferably ≧0.5% and ≦10% by weight of the polyurethane urea as active substance, based on the total mass of the hair-styling composition.

Active substance is understood to mean the polyurethane urea without solvent or solvent mixture.

The hairsetting compositions of the invention are preferably in the form of gels, sprays or aerosols, which are preferably transparent. “Transparent” in the context of the present invention means that the turbidity values of the composition are ≦100 NTU (Nephelometric Turbidity Unit), preferably ≦50 NTU, more preferably ≦10 NTU and most preferably ≦5 NTU. Turbidity values are determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

Preferably, the hairsetting compositions are in the form of transparent aerosols or sprays. The hairsetting compositions of the invention may advantageously be in a pump spray or aerosol packing. They can also advantageously be foamed with a propellant gas.

Accordingly, pump sprays, aerosol packs and foam dispensers containing the composition of the invention are likewise part of the invention.

The hair-styling compositions of the invention preferably have a viscosity of ≧0.5 and ≦20 000 mPas. Compositions in the form of gels more preferably have a viscosity of ≧2000 and ≦20 000 mPas and most preferably of ≧5000 and ≦10 000 mPas. Sprayable compositions for sprays more preferably have a viscosity of ≧0.5 and ≦500 mPas and most preferably of ≧1 and ≦200 mPas.

The viscosities reported are determined by means of rotary viscometry to DIN 53019 at 23° C. with a rotary viscometer from Anton Paar Germany GmbH, Ostfildern, DE, at a shear rate of 10 s⁻¹.

In a preferred embodiment of the invention, the hair-styling composition comprises at least one propellant gas.

Propellant gases suitable for the hair-styling composition of the invention are hydrocarbons such as propane, isobutane and n-butane, and mixtures thereof. Compressed air, hydrofluorocarbons, for example 1,2-difluoromethane (propellant 152A), carbon dioxide, nitrogen, nitrogen dioxide and dimethyl ether, and mixtures of all these gases, may likewise be used. Preferably, dimethyl ether or mixtures of propane and butane are used in the hair-styling composition of the invention.

The propellant gases preferably account for ≧20% and ≦80% by weight, more preferably ≧35% and ≦65% by weight, based on the total weight of the hair-styling composition. Thickeners advantageous for the hair-styling compositions of the invention are:
Crosslinked or non-crosslinked acrylic acid or methacrylic acid homo- or copolymers. These include homopolymers of methacrylic acid or acrylic acid, copolymers of acrylic acid and/or methacrylic acid and monomers derived from other acrylic or vinyl monomers, such as C₁₀₋₃₀ alkyl acrylates, C₁₀₋₃₀ alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and derivatives thereof, carob flour, hyaluronic acid.
Nonionic, anionic, cationic or amphoteric associative polymers, for example based on polyethylene glycols and derivatives thereof, or polyurethanes.
Crosslinked or non-crosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid and methacrylic acid homo- or copolymers, and crosslinked copolymers of 2-acrylamido-2-methylpropanesulfonic acid.

Very particularly advantageous thickeners are xanthan gum, such as the products supplied under the KELTROL and KELZA names by CP Kelco or the products from RHODIA with the RHODOPOL name, and guar gum, such as the products available under the JAGUAR HP105 name from RHODIA.

Very particularly advantageous thickeners are likewise crosslinked homopolymers of methacrylic acid or acrylic acid, which are commercially available from Lubrizol under the CARBOPOL 940, CARBOPOL 941, CARBOPOL 980, CARBOPOL 981, CARBOPOL ETD 2001, CARBOPOL EDT 2050, CARBOPOL 2984, CARBOPOL 5984 and CARBOPOL ULTREZ 10 names, from 3V under the SYNTHALEN K, SYNTHALEN L and SYNTHALEN MS names, and from PROTEX under the MODAREZ V 1250 PX, MODAREZ V2000 PX, VISCARON A1600 PE and VISCARON A700 PE names.

Many particularly advantageous thickeners are additionally crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such polymers are commercially available, for example, from Lubrizol under the CARBOPOL 1342, CARBOPOL 1382, PEMULEN TR1 or PEMULEN TR2 names and from ISP under the ULTRATHIX P-100 (INCI: Acrylic Acid/VP Cross polymer) names.

Very particularly advantageous thickeners are likewise crosslinked copolymers of 2-acrylamido-2-methylpropanesulfonic acid. Such copolymers are available, for example, from Clariant under the ARISTOFLEX AVC names (INCI: Ammonium Acryloyldimethyltaurate/VP Copolymer).

Thickeners are generally used in a concentration of ≧0% and ≦2% by weight, preferably of ≧0.1% and ≦1% by weight.

Haircare substances may additionally be present in the compositions of the invention. Care substances used may preferably be panthenol and/or cyclic polydimethylsiloxanes (cyclomethicones) or silicone surfactants (polyether-modified siloxanes) of the dimethicone copolyol or simethicone type.

Cyclomethicones are supplied, inter alia, under the ABIL K4 trade names by Goldschmidt or, for example, DC 244, DC 245 or DC 345 from Dow Corning. Dimethicone copolyols are supplied, for example, under the DC 193 trade name by Dow Corning or BELSIL DM 6031 by Wacker.

The hair-styling compositions of the invention may, as well as the polyurethane ureas described above, also comprise further suitable film formers which may especially also contribute to setting and to styling of the hair.

The proportion of one or more further film formers may be ≧0% and ≦20% by weight and especially ≧0% and ≦10% by weight, based on the overall formulation.

Advantageously, the further film former(s) is/are selected from the group of the nonionic, anionic, amphoteric and/or cationic polymers and mixtures thereof.

Suitable nonionic polymers which may be present in the composition of the invention alone or in mixtures, preferably also with anionic and/or amphoteric and/or zwitterionic polymers, are selected from the group of:
polyalkyloxazolines,
vinyl acetate homo- or copolymers, including, for example, copolymers of vinyl acetate and acrylic esters, copolymers of vinyl acetate and ethylene, copolymers of vinyl acetate and maleic esters;
acrylic ester copolymers, for example the copolymers of alkyl acrylate and alkyl methacrylate, copolymers of alkyl acrylate and urethanes;
copolymers of acrylonitrile and nonionic monomer selected from butadiene and (meth)acrylate;
styrene homo- and copolymers, including, for example, homopolystyrene, copolymers of styrene and alkyl (meth)acrylate, copolymers of styrene, alkyl methacrylate and alkyl acrylate, copolymers of styrene and butadiene, copolymers of styrene, butadiene and vinylpyridine;
polyamides,
vinyllactam homo- or copolymers, such as vinylpyrrolidone homo- or copolymers, including, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate;
polysiloxanes; and
homopolymers of N-vinylformamide.

Particularly preferred nonionic polymers are acrylic ester copolymers, homopolymers of vinylpyrrolidone and copolymers, and polyvinylcaprolactam.

Very particularly preferred nonionic polymers are homopolymers of vinylpyrrolidone, for example LUVISKOL K from BASF, copolymers of vinylpyrrolidone and vinyl acetate, for example LUVISKOL VA products from BASF or PVPVA S630L from ISP, terpolymers of vinylpyrrolidone, vinyl acetate and propionate, for example LUVISKOL VAP from BASF and polyvinylcaprolactams, for example LUVISKOL PLUS from BASF.

Advantageous anionic polymers are homo- or copolymers with monomer units which contain acid groups and have optionally been copolymerized with comonomers not containing acid groups. Suitable monomers are unsaturated, free-radically polymerizable compounds having at least one acid group, and especially carboxylic acid, sulfonic acid or phosphoric acid.

Examples of anionic polymers containing carboxylic acid groups are:
acrylic acid or methacrylic acid homo- or copolymers or salts thereof. These include, for example, the copolymers of acrylic acid and acrylamide and/or sodium salts thereof, copolymers of acrylic acid and/or methacrylic acid and an unsaturated monomer selected from the group of ethylene, styrene, vinyl esters, acrylic esters, methacrylic esters, optionally ethoxylated compounds, copolymers of vinylpyrrolidone, acrylic acid and C₁-C₂₀ alkyl methacrylate, e.g. ACRYLIDONE LM from ISP, copolymers of methacrylic acid, ethyl acrylate and tert-butyl acrylate, e.g. LUVIMER 100 P from BASF;
crotonic acid derivative homo- or copolymers or salts thereof. These include, for example, vinyl acetate/crotonic acid, vinyl acetate/acrylate and/or vinyl acetate/vinyl neodecanoate/crotonic acid copolymers, for example RESYN 28-1310 or RESYN 28-2930 from AkzoNobel or LUVISET CAN from BASF, sodium acrylate/vinyl alcohol copolymers;
unsaturated C₄-C₈ carboxylic acid derivatives or carboxylic anhydride copolymers selected from copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic ester is or copolymers of maleic acid/maleic anhydride or fumaric acid/fumaric anhydride or itaconic acid/itaconic anhydride and at least one monomer selected from the group of allyl esters, methallyl esters and optionally acrylamides, methacrylamides, α-olefin, acrylic esters, methacrylic esters, vinylpyrrolidones. Further preferred polymers are methyl vinyl ethers/maleic acid copolymers obtained through hydrolysis of vinyl ether/maleic anhydride copolymers. These polymers may also be partly esterified (ethyl, isopropyl or butyl esters) or partly amidated;
water-soluble or -dispersible anionic polyurethanes, e.g. LUVISET PUR from BASF, which are different than the polyurethanes of the invention.

Advantageous anionic polymers containing sulfo groups are salts of polyvinylsulfonic acids, polystyrenesulfonic acids, for example sodium polystyrenesulfonate, or polyacrylamidosulfonic acids.

Particularly advantageous anionic polymers are acrylic acid copolymers, crotonic acid derivative copolymer, copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic esters and salts of polystyrenesulfonic acids.

Very particularly advantageous anionic polymers are acrylate copolymers, e.g. LUVIMER from BASF, ethyl acrylate/N-tert-butylacrylamide/acrylic acid copolymers ULTRAHOLD STRONG from BASF, VA/crotonate/vinyl neodecanoate copolymer, e.g. RESYN 28-2930 from AkzoNobel, copolymers, for example copolymers of methyl vinyl ether and maleic anhydride in partly esterified form, e.g. GANTREZ from ISP, and sodium polystyrenesulfonates, e.g. FLEXAN 130 from AkzoNobel.

Advantageous amphoteric polymers may be selected from the polymers containing units A and B distributed randomly in the polymer chain, where A is a unit derived from a monomer having at least one basic nitrogen atom, and B represents a unit that originates from an acidic monomer having one or more carboxyl or sulfo groups. Alternatively, A and B may be groups derived from zwitterionic carboxybetaine monomers or sulfobetaine monomers. A and B may also be a cationic polymer chain containing primary, secondary, tertiary or quaternary groups, where at least one amino group bears a carboxyl group or sulfo group bonded via a tertiary hydrocarbyl group, or A and B are part of a polymer chain with an ethylene-α,β-dicarboxylic unit in which the carboxylic acid group has been reacted with a polyamine containing one or more primary or secondary amino groups.

Particularly advantageous amphoteric polymers are:
polymers which are formed in the copolymerization of a monomer derived from a vinyl compound having a carboxyl group, such as, more particularly, acrylic acid, methacrylic acid, maleic acid, α-chloroacrylic acid, and a basic monomer. The basic monomer is derived from a compound which is substituted and contains at least one basic atom, such as, more particularly, dialkylaminoalkyl methacrylate and acrylate, dialkylaminoalkylmethacrylamide and -acrylamide. Compounds of this kind have been described in U.S. Pat. No. 3,836,537.
Polymers having units derived from: a) at least one monomer selected from the acrylamides or methacrylamides, substituted by an alkyl group on the nitrogen atom, b) at least one acidic comonomer containing one or more reactive carboxyl groups, and c) at least one basic comonomer, such as esters of acrylic acid and methacrylic acid with primary, secondary, tertiary and quaternary amino substituents and the quaternization product of dimethylaminoethyl methacrylate with dimethyl sulfate or diethyl sulfate.

Particularly preferred N-substituted acrylamides or methacrylamides are compounds wherein the alkyl groups contain 2 to 12 carbon atoms. Very particular preference is given to N-ethylacrylamide, N-t-butylacrylamide, N-t-octylacrylamide, N-octylacrylamide, N-decylacrylamide, N-dodecylacrylamide and the corresponding methacrylamides.

Suitable acidic comonomers are especially selected from the group of acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.

Preferred basic comonomers are aminoethyl methacrylate, butylaminoethyl methacrylate, N,N-dimethylaminoethyl methacrylate, N-t-butylaminoethyl methacrylate.
Crosslinked and wholly or partly acylated polyaminoamides derived from polyaminoamides of the following general formula:

—[CO—R—CO—Z]—

in which R is a divalent group derived from a saturated dicarboxylic acid, an aliphatic mono- or dicarboxylic acid with an ethylenic double bond, an ester of these acids with a lower alkanol having 1 to 6 carbon atoms or a group that forms on addition of one of these acids onto a bis-primary or bis-secondary amine, and Z is a group derived from a bis-primary or mono- to bis-secondary polyalkylene-polyamine.

The saturated carboxylic acids are preferably selected from the acids having 6 to 10 carbon atoms, such as adipic acid, 2,2,4-trimethyladipic acid and 2,4,4-trimethyladipic acid, terephthalic acid; acids having an ethylenic double bond, for example acrylic acid, methacrylic acid and itaconic acid.

The alkane sultones used in the acylation are preferably propane sultone or butane sultone; the salts of acylating agents are preferably the sodium salts or potassium salts.
Polymers having zwitterionic units of the following formula:

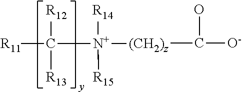

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group chosen such that the sum total of the carbon atoms R₁₄ and R₁₅ does not exceed 10.
Polymers containing such units may also have units that originate from non-zwitterionic monomers, such as dimethyl- and diethylaminoethyl acrylate or dimethyl and diethylaminoethyl methacrylate or alkyl acrylates or alkyl methacrylates, acrylamides or methacrylamides or vinyl acetate.
Polymers which are derived from chitosan and contain monomer units corresponding to the following formulae:

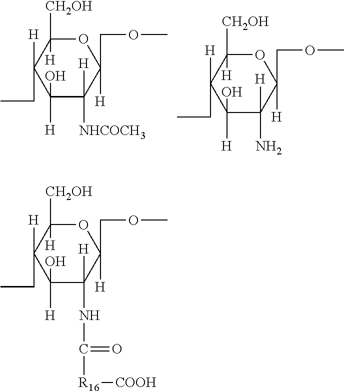

where the first unit is present in proportions of 0% to 30%, the second unit in proportions of 5% to 50% and the third unit in proportions of 30% to 90%, with the proviso that Rib in the third unit is a group of the following formula:

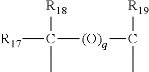

in which, if q=0, the R₁₇, R₁₈ and R₁₉ groups are the same or different and are each a hydrogen atom, methyl, hydroxyl, acetoxy or amino, a monoalkylamine radical or a dialkylamine radical which is optionally interrupted by one or more nitrogen atoms and/or optionally carries one or more amino groups, hydroxyl groups, carboxyl groups, alkylthio groups, sulfo groups, alkylthio groups wherein the alkyl group bears an amino radical, where at least one of the R₁₇, R₁₈ and R₁₉ groups in this case is a hydrogen atom; or, if q=1, the R₁₇, R₁₈ and R₁₉ groups are each a hydrogen atom, and the salts that form these compounds with bases or acids.
Polymers which correspond to the following general formula and are described, for example, in the French patent 1 400 366:

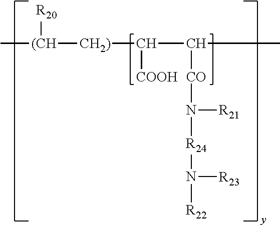

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl, or a group of the formula: —R₂₄—N(R₂₂)₂ where R₂₄ is a —CH₂—CH₂, —CH₂—CH₂—CH₂— or —CH₂—CH(CH₃)— group and where R₂₂ has the definitions given above.
Polymers which can be formed in the N-carboxyalkylation of chitosan, such as N-carboxymethylchitosan or N-carboxybutylchitosan.
Alkyl(C₁₋₅) vinyl ether/maleic anhydride copolymers which have been modified partly by semiamidation with an N,N-dialkylaminoalkylamine such as N,N-dimethylaminopropylamine or an N,N-dialkylamino alcohol. These polymers may also contain further comonomers, such as vinylcaprolactam.

Very particularly advantageous amphoteric polymers are copolymers of at least one monomer having acrylate groups with at least one further comonomer, for example the copolymers of octylacrylamide with t-butylaminomethyl methacrylate, and acrylic acid and/or methacrylic acid and/or esters thereof (e.g. INCI: octylacrylamide/acrylates butylaminoethyl methacrylate copolymer), commercially available under the AMPHOMER, AMPHOMER LV 71 or BALANCE 47 names from AkzoNobel, and methyl methacrylate/methyldimethylcarboxymethylammonioethyl methacrylate copolymers.

It may be advantageous to neutralize the anionic and amphoteric polymers with suitable bases to improve their water solubility or their water dispersibility.

Neutralizing agents used for polymers containing acid groups may be the following bases: hydroxides wherein the cation is ammonium or an alkali metal, for example NaOH or KOH.

Other neutralizing agents are primary, secondary or tertiary amines, amino alcohols or ammonia. Preference is given here to using 2-amino-2-methylpropane-1,3-diol (AMPD), 2-amino-2-ethylpropane-1,3-diol (AEPD), 2-amino-2-methyl-1-propanol (AMP), 2-amino-1-butanol (AB), 2-aminopropane-1,3-diol, monoethanolamine (MEA), diethanolamine (DEA), triethanolamine (TEA), monoisopropanolamine (MIPA), diisopropanolamine (DIPA), triisopropanolamine (TIPA), dimethyl laurylamine (DML), dimethyl myristalamine (DMM), and dimethyl stearamine (DMS).

The neutralization may be partial or complete according to the end use.

It is optionally possible to use, albeit less preferably, cationic polymers, for example polymers containing primary, secondary, tertiary and/or quaternary amino groups that are part of the polymer chain or bonded directly to the polymer chain.

It is optionally possible for additives customary for hair-styling compositions likewise to be present in the composition. These may be, for instance, silicones or silicone derivatives, wetting agents, humectants, plasticizers such as glycerol, glycol and phthalic esters and ethers, UV absorbers, dyes, pigments and other colorants, anticorrosive agents, neutralizing agents, antisticking agents, combining agents and conditioners, antistats, shine agents, preservatives, proteins and derivatives thereof, amino acids, vitamins, emulsifiers, surface-active agents, viscosity modifiers, thickeners and rheology modifiers, gelating agents, stabilizers, surfactants, sequestrants, complexing agents, pearlescent agents, esthetic enhancers, fatty acids, fatty alcohols, triglycerides, botanic extracts and clarifying aids.

These additives may be present in a total concentration of about ≧0% and ≦15% by weight, preferably ≧0.01% and ≦10% by weight, based on the total weight of the composition.

In a preferred embodiment of the invention, the hairstyling composition comprises
A) at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
B) at least one propellant selected from dimethyl ether and a mixture of propane and butane,
C) at least one aliphatic alcohol having 1 to 6 carbon atoms,
D) haircare substances and
E) optionally, further film formers.

In a particularly preferred embodiment of the invention, the hair-styling composition comprises
A) ≧0.5% and ≦80% by weight of at least one polyurethane urea dissolved in a solvent or solvent mixture, where the solvent consists of one or more monohydroxy-functional alcohols or a solvent mixture consisting of organic solvents containing ≧50% by weight, based on the total mass of the solvent mixture, of at least one monohydroxy-functional alcohol is used,
B) ≧35% and ≦65% by weight of at least one propellant selected from dimethyl ether and/or a mixture of propane and butane,
C) ≧10% and ≦90% by weight, based on the overall composition, of at least one aliphatic alcohol having 1 to 6 carbon atoms,
D) ≧0.01% and ≦10% by weight of haircare substances and
E) ≧0% and ≦20% of further film formers, based in each case on the total mass of the hair-styling composition, where components A) to E) add up to 100% by weight.

The solids content of the polyurethane urea solution is preferably chosen such that the hair-styling compositions contain ≧0.2% and ≦20% by weight of the polyurethane urea as active substance, based on the total mass of the hair-styling composition. In a particularly advantageous embodiment, the hair-styling composition contains a further film former E), especially in a proportion of ≧1% and ≦20% by weight, preferably ≧1% and ≦10% by weight.

The further film former E) particularly advantageously comprises copolymers of at least one monomer having acrylate groups with at least one further comonomer, for example the copolymers of copolymers of octylacrylamide with t-butylaminomethyl methacrylate, and acrylic acid and/or methacrylic acid and/or esters thereof (e.g. INCI: octylacrylamide/acrylates butylaminoethyl methacrylate copolymer), commercially available under the AMPHOMER, AMPHOMER LV 71 or BALANCE 47 names from AkzoNobel, and/or methyl methacrylate/methyldimethylcarboxymethylammonioethyl methacrylate copolymers.

Suitable monomers having acrylate groups are especially methyl acrylate, ethyl acrylate, propyl acrylate, isopropyl acrylate, n-butyl acrylate, sec-butyl acrylate, tert-butyl acrylate, pentyl acrylate, hexyl acrylate, heptyl acrylate, octyl acrylate, 2-octyl acrylate, ethylhexyl acrylate, nonyl acrylate, 2-methyloctyl acrylate, 2-tert-butylheptyl acrylate, 3-isopropylheptyl acrylate, decyl acrylate, undecyl acrylate, 5-methylundecyl acrylate, dodecyl acrylate, 2-methyldodecyl acrylate, tridecyl acrylate, 5-methyltridecyl acrylate, tetradecyl acrylate, pentadecyl acrylate, hexadecyl acrylate, 2-methylhexadecyl acrylate, heptadecyl acrylate, 5-isopropylheptadecyl acrylate, 5-ethyloctadecyl acrylate, octadecyl acrylate, nonadecyl acrylate, eicosyl acrylate, cycloalkyl acrylates, for example cyclopentyl acrylate, cyclohexyl acrylate, 3-vinyl-2-butylcyclohexyl acrylate, cycloheptyl acrylate, cyclooctyl acrylate, bornyl acrylate, tetrahydrofurfuryl acrylate and isobornyl acrylate. Preference is given to ethyl acrylate, n-butyl acrylate, ethylhexyl acrylate, cyclohexyl acrylate and particular preference to ethyl acrylate, n-butyl acrylate or ethylhexyl acrylate.

Advantageously, the further monomer is selected from the group of the nonionic, anionic, amphoteric and/or cationic monomer or mixtures thereof.

Particularly suitable further monomers, which may be present alone or in mixtures, preferably also with anionic and/or amphoteric and/or zwitterionic monomers, are:
styrene or derivatives thereof,
vinyl acetate,
acrylic acid or methacrylic acid or salts thereof. These include, for example, copolymers of vinylpyrrolidone, acrylic acid and C₁-C₂₀ alkyl methacrylates, e.g. ACRYLIDONE LM from ISP, copolymers of methacrylic acid, ethyl acrylate and tert-butyl acrylate, e.g. LUVIMER 100 P from BASF, ethyl acrylate/N-tert-butylacrylamide/acrylic acid copolymers ULTRAHOLD STRONG from BASF;
crotonic acid derivative or salts thereof. These include, for example, vinyl acetate/crotonic acid, vinyl acetate/acrylate and/or vinyl acetate/vinyl neodecanoate/crotonic acid copolymers, for example RESYN 28-1310 or RESYN 28-2930 from AkzoNobel or LUVISET CAN from BASF, sodium acrylate/vinyl alcohol copolymers;
unsaturated C₄-C₈ carboxylic acid derivatives or carboxylic anhydride copolymers selected from copolymers of maleic acid/maleic anhydride or fumaric acid/fumaric anhydride or itaconic acid/itaconic anhydride and at least one acrylic acid ester. Very particularly advantageous polymers are copolymers of methyl vinyl ether and maleic anhydride in partly esterified form, e.g. GANTREZ;
acrylamides or methacrylamides. Examples of these include acrylates/octylacrylamide copolymer, e.g. AMPHOMER HC from AkzoNobel;
basic monomers derived from a substituted vinyl compound having at least one basic atom, such as, more particularly, dialkylaminoalkyl methacrylate and acrylate, dialkylaminoalkyl(meth)acrylamide and -acrylamide. Preferred basic comonomers are aminoethyl methacrylate, butylaminoethyl methacrylate, N,N-dimethylaminoethyl methacrylate, N-t-butylaminoethyl methacrylate.

It is most preferable when the copolymer comprises or consists of octylacrylamide/acrylates/butylaminoethyl methacrylate copolymer.

The invention further provides for the use of the hair-styling composition of the invention for shaping, setting and/or fixing hair.

The invention likewise provides a method of shaping, setting or fixing hair using the hair-styling compositions of the invention, wherein the hair-styling composition is applied to the hair.

Advantageously, in the method of the invention, the hair-styling compositions of the invention remain at least partly on the hair.

The present invention is elucidated by the following examples.

### EXAMPLES

Unless indicated otherwise, all percentages are based on weight.

Unless stated otherwise, all analytical measurements relate to temperatures of 23° C. The solids contents (nonvolatile component) were determined to DIN EN ISO 3251. Unless explicitly mentioned otherwise, NCO contents were determined by volumetric means to DIN EN ISO 11909.

The check for free NCO groups was conducted by means of IR spectroscopy (band at 2260 cm⁻¹).

The viscosities reported were determined by means of rotary viscometry to DIN 53019 at 23° C. with a rotary viscometer from Anton Paar Germany GmbH, Ostfildern, DE.

The number-average molecular weight was determined by gel permeation chromatography (GPC) in tetrahydrofuran at 23° C. The procedure is according to DIN 55672-1: “Gel permeation chromatography, Part 1—Tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used for calibration. The number-average molecular weight is calculated with software support. Baseline points and evaluation limits are fixed in accordance with DIN 55672 Part 1.

Turbidity values [NTU] were determined by a scattered light measurement at a 90° angle (nephelometry) at a measurement radiation wavelength of 860 nm in accordance with DIN EN ISO 7027, conducted at 23° C. with a model 2100AN laboratory turbidimeter from HACH LANGE GmbH, Berlin, Germany.

### Substances and Abbreviations Used:

POLYTHF 2000: polytetramethylene glycol polyol, OH number 56 mg KOH/g, number-average molecular weight 2000 g/mol (BASF AG, Ludwigshafen, DE)
POLYTHF 1000: polytetramethylene glycol polyol, OH number 112 mg KOH/g, number-average molecular weight 1000 g/mol (BASF AG, Ludwigshafen, DE)
Ethanol unless stated otherwise, MEK-denatured ethanol from Nordbrand, Nordhausen, DE was used.

Isocyanates and the further polymeric polyols were used from Covestro AG (formerly Bayer MaterialScience AG), Leverkusen, DE. Further chemicals were purchased from Sigma-Aldrich Chemie GmbH, Taufkirchen, DE. The raw materials, unless stated otherwise, were used without further purification or pretreatment.

### Example 1: Preparation of a Polyurethane Solution in Ethanol (Inventive)

150 g of POLYTHF 2000 and 37.50 g of POLYTHF 1000 were dewatered under membrane pump vacuum at 100° C. for one hour in a standard stirrer apparatus and then initially charged at 80° C. under nitrogen. Then 75.06 g of isophorone diisocyanate were added at 80° C. within 5 min and stirring at 110° C. was continued (about 3 hours) until the NCO value had gone below the theoretical value. The prepolymer was cooled to 40° C. and it was dissolved in 630.4 g of ethanol (denatured with diethyl phthalate) and then the temperature was reduced to 15° C. Then a solution of 37.6 g of methylenebis(4-aminocyclohexane) and 270 g of ethanol (denatured with diethyl phthalate) was metered in within 30 min; after a further 30 minutes at 20° C., isocyanate groups were still detectable by IR spectroscopy. Stirring of the mixture was continued at 23° C. for about 16 hours until no free isocyanate groups were detectable any longer by IR spectroscopy.

The resultant clear, storage-stable solution had the following properties:

Solids content: 23%
Viscosity (viscometer, 23° C.): 280 mPas
Turbidity value: 1.2 NTU

### Example 2: Preparation of a Polyurethane Solution in Ethanol (Inventive)

300 g of POLYTHF 1000 were dewatered under membrane pump vacuum at 100° C. for one hour in a standard stirrer apparatus and then initially charged at 80° C. under nitrogen. Then 133.44 g of isophorone diisocyanate were added at 80° C. within 5 min and stirring at 110° C. was continued (about 3 hours) until the NCO value had gone below the theoretical value. The prepolymer was cooled to 40° C. and it was dissolved in 517 g of ethanol (denatured with MEK) and then the temperature was reduced to 16° C. Then a solution of 58.8 g of methylenebis(4-aminocyclohexane) and 222 g of ethanol (denatured with MEK) was metered in within 30 min; then a further 410 g of ethanol were added. Stirring was continued until no free isocyanate groups were detectable any longer by IR spectroscopy.

The resultant clear, storage-stable solution had the following properties:

Solids content: 30.2%
Viscosity (viscometer, 23° C.): 85 000 mPas

### Example 3: Preparation of a Polyurethane Solution in Ethanol (Inventive)

300 g of POLYTHF 1000 were dewatered under membrane pump vacuum at 100° C. for one hour in a standard stirrer apparatus and then initially charged at 80° C. under nitrogen. Then 133.44 g of isophorone diisocyanate were added at 80° C. within 5 min and stirring at 110° C. was continued (about 3 hours) until the NCO value had gone below the theoretical value. The prepolymer was cooled to 40° C. and it was dissolved in 498 g of ethanol (denatured with MEK) and then the temperature was reduced to 16° C. Then a solution of 40.9 g of isophoronediamine and 213 g of ethanol (denatured with MEK) was metered in within 30 min; then a further 7.15 g of ethanol isophoronediamine were added. Stirring was continued until no free isocyanate groups were detectable any longer by IR spectroscopy.

The resultant clear, storage-stable solution had the following properties:

Solids content: 40.8%
Viscosity (viscometer, 23° C.): 3850 mPas

### Example 4: Preparation of a Polyurethane Solution in Ethanol (Inventive)

250 g of POLYTHF 2000 and 62.5 g of POLYTHF 1000 were dewatered under membrane pump vacuum at 100° C. for one hour in a standard stirrer apparatus and then initially charged at 80° C. under nitrogen. Then 83.4 g of isophorone diisocyanate were added at 80° C. within 5 min and stirring at 110° C. was continued (about 3 hours) until the NCO value had gone below the theoretical value. The prepolymer was cooled to 40° C. and it was dissolved in 425 g of ethanol and then the temperature was reduced to 18° C. Then a solution of 9.0 g of ethylenediamine and 180 g of ethanol was metered in within 30 min. A further 0.83 g of ethylenediamine was added and then stirring was continued until no free isocyanate groups were detectable any longer by IR spectroscopy.

The resultant clear, storage-stable solution had the following properties:

Solids content: 40.7%
Viscosity (viscometer, 23° C.): 34600 mPas

### Example 5: Preparation of a Polyurethane Solution in Ethanol (Inventive)

211 g of POLYTHF 2000 and 52.7 g of POLYTHF 1000 were dewatered under membrane pump vacuum at 100° C. for one hour in a standard stirrer apparatus, then 5.4 g of neopentyl glycol were added and the mixture was subsequently initially charged at 80° C. under nitrogen. Then 93.4 g of isophorone diisocyanate were added at 80° C. within 5 min and stirring at 110° C. was continued (about 3 hours) until the NCO value had gone below the theoretical value. The prepolymer was cooled to 40° C. and it was dissolved in 420 g of ethanol (denatured with diethyl phthalate) and then the temperature was reduced to 17° C. Then a solution of 35.3 g of methylenebis(4-aminocyclohexane) and 180 g of ethanol (denatured with diethyl phthalate) was metered in within 30 min. A further 0.67 g of methylenebis(4-aminocyclohexane) were added and then stirring was continued until no free isocyanate groups were detectable any longer by IR spectroscopy.

The resultant clear, storage-stable solution had the following properties:

Solids content: 40.5%
Viscosity (viscometer, 23° C.): 7060 mPas

### Example 6: Preparation of a Polyurethane Solution in Ethanol (Inventive)

226.2 g of polypropylene glycol having a number-average molecular weight of 2000 g/mol and 62.5 g of polypropylene glycol having a number-average molecular weight of 1000 g/mol were dewatered under membrane pump vacuum at 100° C. for one hour in a standard stirrer apparatus, then the mixture was initially charged at 80° C. under nitrogen. Then 83.4 g of isophorone diisocyanate were added at 80° C. within 5 min and the mixture was stirred at 120° C. for 6 hours until the NCO value had gone below the theoretical value. The prepolymer was cooled to 40° C. and it was dissolved in 280 g of ethanol and then the temperature was reduced to 18° C. Then a solution of 34.1 g of methylenebis(4-aminocyclohexane) and 120 g of ethanol was metered in within 30 min. A further 4.5 g of methylenebis(4-aminocyclohexane) were added and then stirring was continued until no free isocyanate groups were detectable any longer by IR spectroscopy.

The resultant clear, storage-stable solution had the following properties:

Solids content: 49.8%
Viscosity (viscometer, 23° C.): 1100 mPas

### Example 7: Preparation of a Polyurethane Solution in Ethanol (Inventive)

160 g of POLYTHF 2000 and 40.0 g of POLYTHF 1000 were dewatered under membrane pump vacuum at 100° C. for one hour in a standard stirrer apparatus and then initially charged at 80° C. under nitrogen. Then 62.9 g of bis(4,4′-isocyanatocyclohexyl)methane were added at 80° C. within 5 min and stirring at 110° C. was continued (about 3 hours) until the NCO value had gone below the theoretical value. The prepolymer was cooled to 40° C. and it was dissolved in 595 g of ethanol and then the temperature was reduced to 19° C. Then a solution of 20.2 g of methylenebis(4-aminocyclohexane) and 255 g of ethanol was metered in within 30 min. A further 4.5 g of methylenebis(4-aminocyclohexane) were added and then stirring was continued until no free isocyanate groups were detectable any longer by IR spectroscopy.

The resultant clear, storage-stable solution had the following properties:

Solids content: 25.2%
Viscosity (viscometer, 23° C.): 3400 mPas

### Example 8: Preparation of a Polyurethane Solution in Ethanol (Inventive)

200 g of POLYTHF 2000 and 50.0 g of POLYTHF 1000 were dewatered under membrane pump vacuum at 100° C. for one hour in a standard stirrer apparatus and then initially charged at 70° C. under nitrogen. Then 50.4 g of hexamethylene diisocyanate were added at 70° C. within 5 min and stirring at 110° C. was continued (about 3 hours) until the NCO value had gone below the theoretical value. The prepolymer was cooled to 40° C. and it was dissolved in 340 g of ethanol and then the temperature was reduced to 18° C. Because of the high viscosity, a further 270 g of ethanol were added, then a solution of 25.2 g of methylenebis(4-aminocyclohexane) and 150 g of ethanol was metered in within 30 min. Then stirring was continued until it was no longer possible to detect any free isocyanate groups by IR spectroscopy.

The resultant clear, storage-stable solution had the following properties:

Solids content: 30.1%
Viscosity (viscometer, 23° C.): 15500 mPas

### Comparative Example 1: Attempted Preparation of a Polyurethane Urea Solution in Ethanol

100 g of POLYTHF 2000, 25.0 g of POLYTHF 1000 and 127.5 g of a linear difunctional amorphous polyester diol based on adipic acid, hexane-1,6-diol and neopentyl glycol and having a number-average molecular weight of 1700 g/mol were dewatered under membrane pump vacuum at 100° C. for one hour in a standard stirrer apparatus and then initially charged at 80° C. under nitrogen. Then 66.7 g of isophorone diisocyanate were added at 80° C. within 5 min and stirring at 110° C. was continued (about 3 hours) until the NCO value had gone below the theoretical value. The prepolymer was cooled to 40° C. and it was dissolved in 720 g of ethanol, although the product did not dissolve completely, and then the temperature was reduced to 17° C. Then a solution of 25.2 g of methylenebis(4-aminocyclohexane) and 310 g of ethanol was metered in within 30 min, which gave rise to white turbidity. Then stirring was continued, which did not form a stable solution but resulted in a biphasic mixture from which the solid phase settled out.

### Comparative Example 2: Attempted Preparation of a Polyurethane Urea Solution in Ethanol

200 g of a linear difunctional polycarbonate diol based on hexane-1,6-diol, having a number-average molecular weight of 2000 g/mol, and 50 g of a linear difunctional polycarbonate diol based on hexane-1,6-diol, having a number-average molecular weight of 1000 g/mol, were dewatered under membrane pump vacuum at 100° C. for one hour in a standard stirrer apparatus and then initially charged at 80° C. under nitrogen. Then 66.7 g of isophorone diisocyanate were added at 80° C. within 5 min and stirring at 110° C. was continued (about 3 hours) until the NCO value had gone below the theoretical value. The prepolymer was cooled to 40° C. and it was dissolved in 720 g of ethanol, although the product did not dissolve completely, and then the temperature was reduced to 17° C. Then a solution of 25.2 g of methylenebis(4-aminocyclohexane) and 310 g of ethanol was metered in within 30 min, which gave rise to a biphasic mixture. Then stirring was continued, which did not form a stable solution but resulted in a biphasic mixture from which the solid phase settled out.

Performance Tests: Compatibility with Propane/Butane Propellant Mixture

A mixture of propane and butane (weight ratio 2:1) was mixed with concentrations according to table 1 with a polyurethane urea solution of the invention or a polyurethane urea dispersion according to the state of the art, each containing 2% by weight of polyurethane urea, with a stirrer at room temperature for about 10 min. The stability and appearance were each determined directly after mixing at room temperature. Table 1 summarizes the results and shows that stable and transparent mixtures are obtained only with the polyurethane urea of the invention.

TABLE 1

| | | BAYCUSAN C 1008 | |
|---|---|---|---|
| | | (aqueous, ionically | |
| Concentration of | Concen- | hydrophilizing | |
| propane/butane | tration of | polyurethane dispersion; | |
| propellant | film former | Covestro AG, formerly | Inventive |
| mixture | (solution or | Bayer MaterialScience | polyurethane |
| [% by wt.] | dispersion) | AG, Polyurethane-25) | urea solution |
| 10 | 90 | Slightly turbid | Transparent |
| 20 | 80 | Turbid | Transparent |
| 30 | 70 | Milky | Transparent |
| 40 | 60 | 2 phases | Transparent |
| 50 | 50 | 2 phases | Transparent |

### Curl Retention:

For the curl retention tests, commercially available European mixed hair (useful length: 18 cm, Kerling, weight: 0.7 g) was used. The hair was subjected to a standardized wash procedure prior to use. For this purpose, the hair that had been softened in water for 15 minutes was shampooed with 0.2 mL of standard shampoo for one minute, rinsed thoroughly with warm water, blown dry with cold air and conditioned at 21±1° C. and 50±5% relative humidity. 0.1 g of a solution containing 2% by weight of polymeric active substance (polyacrylate and/or polyurethane urea) was applied to the strands. Strands of the hair thus prepared were wound onto 16 mm rollers and then conditioned at 21±1° C. and 50±5% relative humidity for at least 18 hours.

The solutions used contained the polyurethane urea solution of the invention and/or the octylacrylamide/acrylate/butylaminoethyl methacrylate copolymer product (AMPHOMER from AkzoNobel), 100% neutralized with aminomethylpropanediol and dissolved in ethanol, in the mixture specified in the figures.

The curl retention tests were conducted in a specific climate-controlled chamber at a relative humidity of 90±5%. The temperature in the chamber was 21±1° C. The strands prepared were hung simultaneously in the chamber. The length of the strands was read off on a scale at different times. Each test was conducted with eight strands.

The curl retention was calculated by the following formula:

CR=(l_{f}−lₜ)/(l_{f}−lₜᵢ)×100;

in which l_{f} was the strand length, lₜᵢ the original length of the curled hair strands and lₜ the length of the curled hair strands at time t.

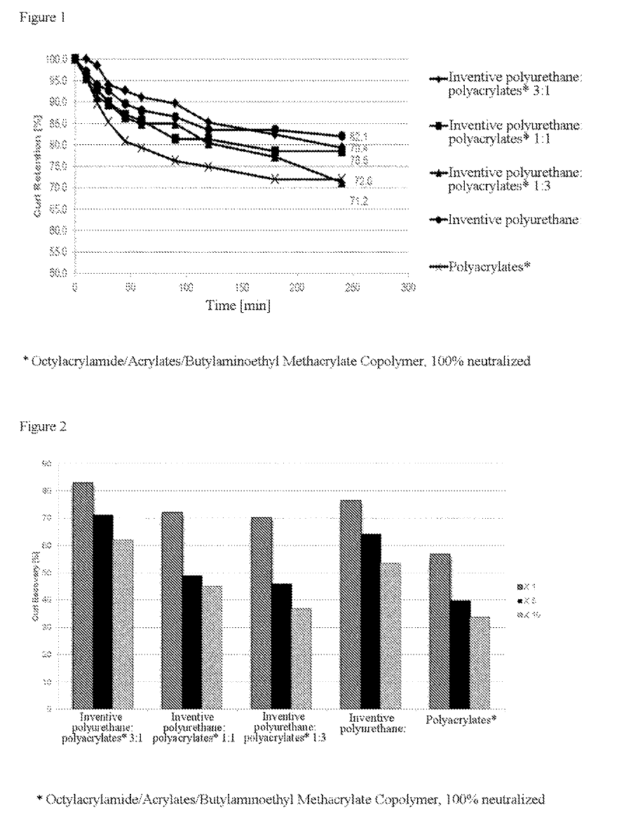

The result of the measurement are shown in the form of a graph in FIG. 1. It is clearly apparent from these that good curl retention is achieved with the polyurethane urea used in accordance with the invention, especially also in combination with polyacrylate film formers according to the state of the art.

For the snap tests, commercially available European mixed hair (useful length: 18 cm, Kerling, weight: 0.7 g) was used. The hair was subjected to a standardized wash procedure prior to use. For this purpose, the hair that had been softened in water for 15 minutes was shampooed with 0.2 mL of standard shampoo for one minute, rinsed thoroughly with warm water, blown dry with cold air and conditioned at 21±1° C. and 50±5% relative humidity.

3 mg of active substance (polyurethane urea used in accordance with the invention or polyacrylate dissolved in ethanol)/1 g of hair strands were applied to the strands by means of a pipette. Fingers were used to distribute the liquid homogeneously over the hair.

The solutions used contained the polyurethane urea solution of the invention and/or the octylacrylamide/acrylate/butylaminoethyl methacrylate copolymer product (AMPHOMER from AkzoNobel), 100% neutralized with aminomethylpropanediol and dissolved in ethanol, in the mixture specified in the figures.

Strands of the hair thus prepared were wound onto 16 mm rollers and then conditioned at 21±1° C. and 50±5% relative humidity for at least 18 hours.

The unwound strands are suspended on an apparatus and the starting length is ascertained with the aid of the scale.

Fingers were used to open up the curls and the length of the strands after time t was determined.

The curl recovery was calculated by the following formula:

CR=(lₜ/lₜᵢ)×100;

in which lₜᵢ was the original length of the curled hair strands and lₜ the length of the curled hair strands at time t.

The test was repeated 10 times.

FIG. 2 also shows that good curl recovery is achieved with the polyurethane urea used in accordance with the invention, especially also in combination with polyacrylate film formers according to the state of the art. “×1” in FIG. 2 means combing the treated hair once; “×5” means combing five times; and “×10” means combing ten times.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. §112(a), and 35 U.S.C. §132(a).

Various aspects of the subject matter described herein are set out in the following numbered clauses:

1. A hairstyling composition comprising at least one propellant gas and/or a thickener, characterized in that it further comprises a polyurethane urea which has no ionically hydrophilizing groups and has been dissolved in a solvent or solvent mixture, the solvent consisting of one or more monohydroxy-functional alcohols or a solvent mixture consisting of organic solvents and containing ≧50% by weight, based on the total mass of the solvent mixture, of at least one monohydroxy-functional alcohol, being used, where the polyurethane urea has been formed from a) at least one aliphatic, araliphatic and/or cycloaliphatic diisocyanate, b) at least one polyether polyol having a number-average molecular weight Mₙ of ≧400 and ≦6000 g/mol and a hydroxyl functionality of ≧1.5 and ≦4, c) at least one amino-functional compound having at least two isocyanate-reactive amino groups, d) optionally at least one alcohol having at least two hydroxyl groups and a molar mass of ≧60 and ≦399 g/mol, e) optionally, at least one compound having a group reactive toward isocyanate groups and f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≧4.

2. The hairstyling composition as in clause 1, characterized in that the polyurethane urea does not have any hydrophilizing groups.

3. The hairstyling composition as in clause 1 or 2, characterized in that component b) is selected from poly(tetramethylene glycol) polyether polyols.

4. The hairstyling composition as in any of clauses 1 to 3, characterized in that component b) has a number-average molecular weight Mₙ of ≧500 and ≦2500 g/mol and a hydroxyl functionality of ≧1.9 and ≦3.

5. The hairstyling composition as in any of clauses 1 to 4, characterized in that component a) is selected from aliphatic, araliphatic and/or cycloaliphatic diisocyanates having at least one isocyanate group bonded to a tertiary carbon atom.

6. The hairstyling composition as in any of clauses 1 to 5, characterized in that component a) is selected from IPDI and/or H12-MDI.

7. The hairstyling composition as in any of clauses 1 to 6, characterized in that component c) is selected from amines having at least two amino groups bonded to primary and/or secondary carbon atoms.

8. The hairstyling composition as in any of clauses 1 to 7, characterized in that the monohydroxy-functional alcohols are selected from aliphatic alcohols having one to six carbon atoms.

9. The hairstyling composition as in any of clauses 1 to 8, characterized in that it contains ≧0.2 and ≦20% by weight of the polyurethane urea as active substance, based on the total weight of the hairstyling composition.

10. The hairstyling composition as in any of clauses 1 to 9, characterized in that it is in the form of an aerosol, spray or gel.

11. The hairstyling composition as in any of clauses 1 to 10, characterized in that it further comprises a further film former which is a copolymer of at least one monomer having acrylate groups with at least one further comonomer.

12. The hairstyling composition as in clause 11, characterized in that the copolymer is Octylacrylamide/Acrylates/Butylaminoethyl Methacrylate Copolymer.

13. The use of the hairstyling composition as in any of clauses 1 to 12 for shaping, setting and/or fixing of hair.

14. A method of shaping, fixing or setting hair using hairstyling compositions as in any of clauses 1 to 12, wherein the hairstyling composition is applied to the hair.

15. A process for producing a hairstyling composition, characterized in that at least one polyurethane urea which has no ionically hydrophilizing groups and has been dissolved in a solvent or solvent mixture is used, the solvent consisting of one or more monohydroxy-functional alcohols or being a solvent mixture consisting of organic solvents and containing ≧50% by weight, based on the total mass of the solvent mixture, of at least one monohydroxy-functional alcohol, wherein the polyurethane urea has been formed from a) at least one aliphatic, araliphatic and/or cycloaliphatic diisocyanate, b) at least one polyether polyol having a number-average molecular weight Mₙ of ≧400 and ≦6000 g/mol and a hydroxyl functionality of ≧1.5 and ≦4, c) at least one amino-functional compound having at least two isocyanate-reactive amino groups, d) optionally, at least one alcohol having at least two hydroxyl groups and a molar mass of ≧60 and ≦399 g/mol, e) optionally, at least one compound having a group reactive toward isocyanate groups and f) optionally, ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4.

1. A hair-styling composition comprising:
at least one of a propellant gas and a thickener, and
a polyurethane urea formed from
a) at least one of an aliphatic, araliphatic and cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally at least one alcohol having at least two hydroxyl groups and a molar mass of ≧60 and ≦399 g/mol,
e) optionally at least one compound having a group reactive toward isocyanate groups and
f) optionally ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≧4
wherein the polyurethane urea contains no ionically hydrophilizing groups and is dissolved in a solvent or solvent mixture, the solvent consisting of one or more monohydroxy-functional alcohols and the solvent mixture consisting of organic solvents and containing ≧50% by weight, based on the total mass of the solvent mixture, of at least one monohydroxy-functional alcohol.

2. The hair-styling composition according to claim 1, wherein the polyurethane urea does not have any hydrophilizing groups.

3. The hair-styling composition according to claim 1, wherein component b) is a poly(tetramethylene glycol) polyether polyol.

4. The hair-styling composition according to claim 1, wherein component b) has a number-average molecular weight Mₙ of ≧500 and ≦2500 g/mol and a hydroxyl functionality of ≧1.9 and ≦3.

5. The hair-styling composition according to claim 1, wherein component a) is selected from the group consisting of aliphatic, araliphatic and cycloaliphatic diisocyanates having at least one isocyanate group bonded to a tertiary carbon atom.

6. The hair-styling composition according to claim 1, wherein component a) is selected from the group consisting of isophorone diisocyanate (IPDI) and H12-MDI.

7. The hair-styling composition according to claim 1, wherein component c) is selected from the group consisting of an amines having at least two amino groups bonded to a primary carbon atom and an amine having at least two amino groups bonded to a secondary carbon atom.

8. The hair-styling composition according to claim 1, wherein the one or more monohydroxy-functional alcohols are aliphatic alcohols having one to six carbon atoms.

9. The hair-styling composition according to claim 1, wherein the composition contains ≧0.2 and ≦20% by weight of the polyurethane-urea, based on the total weight of the hair-styling composition.

10. The hair-styling composition according to claim 1, wherein the composition is in the form of an aerosol, a spray, or a gel.

11. The hair-styling composition according to claim 1, further comprising a further film former which is a copolymer of at least one monomer having acrylate groups with at least one further comonomer.

12. The hair: styling composition according to claim 1, wherein the copolymer is a octylacrylamide/acrylates/butylaminoethyl methacrylate copolymer.

13. (canceled)

14. A method for one of shaping, fixing and setting hair comprising applying to the hair, the hair-styling compositions according to claim 1.

15. A process for producing a hair-styling composition, comprising dissolving in a solvent or a solvent mixture at least one polyurethane urea formed from
a) at least one of an aliphatic, araliphatic and cycloaliphatic diisocyanate,
b) at least one polyether polyol having a number-average molecular weight Mₙ of ≧400 and ≦6000 g/mol and a hydroxyl functionality of ≧1.5 and ≦4,
c) at least one amino-functional compound having at least two isocyanate-reactive amino groups,
d) optionally at least one alcohol having at least two hydroxyl groups and a molar mass of ≧60 and ≦399 g/mol,
e) optionally at least one compound having a group reactive toward isocyanate groups and
f) optionally ≦20% by weight, based on the total mass of the polyurethane urea, of at least one different polyol than b) having a number-average molecular weight Mₙ of ≧500 and ≦6000 g/mol and a hydroxyl functionality of ≧1.5 and ≦4,
wherein the polyurethane urea contains no ionically hydrophilizing groups, and wherein the solvent consists of one or more monohydroxy-functional alcohols or a solvent mixture consisting of organic solvents and containing ≧50% by weight, based on the total mass of the solvent mixture, of at least one monohydroxy-functional alcohol.
